(19) United States
(12) Patent Application Publication
(10) Pub. No.: US 2025/0363447 A1
(43) Pub. Date: Nov. 27, 2025

(54) SYSTEM AND METHOD FOR DISTRIBUTING PERISHABLE ITEMS

(71) Applicant: IDEAL BISTRO LLC, Ridgewood, NY (US)

(72) Inventor: Andrzej Nartowicz, Bialystok (PL)

(21) Appl. No.: 19/297,172

(22) Filed: Aug. 12, 2025

Related U.S. Application Data
(63) Continuation-in-part of application No. 17/610,379, filed on Nov. 10, 2021.
(60) Provisional application No. 62/846,437, filed on May 10, 2019.

(51) Int. Cl. G06Q 10/0836; A61L 9/014; A61L 9/015; A61L 9/16; G06Q 10/0832; G06Q 20/18; G06Q 30/0601; G07F 17/00

(52) U.S. Cl. CPC G06Q 10/08365; A61L 9/014; A61L 9/015; A61L 9/16; G06Q 10/0832; G06Q 20/18; G06Q 30/0633; G06Q 30/0641; G07F 17/0064; A61L 2209/14; A61L 2209/212; A61L 2209/22

(57) ABSTRACT

A unified food distribution ecosystem is described. In one version, it includes a food dispensing unit, a return module, a beverage dispenser, and a refrigerated vending unit, all interconnected via a centralized mobile application and cloud-based management platform. Each module shares user identity management, payment systems, and AI-driven personalization features to deliver a seamless food service experience.

This application is a continuation in part of U.S. application Ser. No. 17/610,379, filed on Nov. 10, 2021, presently pending, which in turn claimed priority to PCT Application PCT/IB2020/054401, filed on May 9, 2020, presently expired, which in turn claimed priority to U.S. Provisional Application 62/846,437, filed on May 10, 2019, the contents of which are incorporated by reference.

### BACKGROUND OF THE INVENTION

### 1. Field of the Invention

The field of the invention is an integrated system that facilitates the distribution of perishable items, along with a method of using the system to distribute items to customers efficiently.

### 2. Background of the Invention

In various embodiments, the invention provides a system and method for the distribution of an inventory to end-users.

In one embodiment, the invented system comprises several logical and physical layers. At one level, the end customer interacts with the system by placing requests for items, such as by using an appropriate app on a special-purpose or general computing device. The requests are forwarded to a distribution network that delivers the requested item to a designated distribution point. The requesting user then retrieves the item at the distribution point, being able to select both the destination location and a time of retrieval. The network delivery points comprise another layer of the system, as does the logical network of suppliers fulfilling orders at the delivery points.

In prior art approaches, a single entity would attempt to fulfill most or all functions within the distribution network. For example, traditional vending machine owners are commonly vertically integrated with the wholesale distributors supplying the machines. Prior art approaches create inefficiency and prevent innovative products from being brought to market, as every wholesaler seeks to control every aspect of distribution. In such a system, introducing a new product, such as perishable foods, has significant barriers to entry, such as the requirement to build out a distribution network.

A need exists in the art for a system and method of distribution of products to end-users that allows for cooperation throughout the distribution network and allows for a variety of products to be ordered and distributed.

### SUMMARY OF INVENTION

An object of the invention is to create a system and method of distributing products to customers. An advantage of the invention is that it allows for a variety of products to be distributed by a diverse set of suppliers.

Another object of the invention is to facilitate the distribution of perishable items. A feature of the invention is that the distribution points include environmental controls. An advantage of the invention is that it allows for the safe distribution of temperature and time-sensitive perishable items, such as food.

Yet another object of the invention is to provide a system and method supporting just-in-time delivery. A feature of the invention is that the end customer can specify a pickup time for an order at a pickup point. An advantage of one embodiment is that the supplier of the perishable items can ensure the freshness of the items at time of delivery and consumption.

A further object of the invention is to provide a system for tracking order status. A feature of the invention is that in one embodiment, each distribution point includes an accounting of the contents of the distribution point, including loading and unloading times. An advantage of the system is that it supports the gathering of information about customer requests and consumption needs.

Another object of the invention is to provide a means for suppliers to enter new markets. A feature of the invention, in one embodiment, is that independent suppliers can provision the distribution points based on real-time demand. A benefit of the invention is that a supplier can enter a market without building out a network of warehouses and instead can use the existing distribution points.

An additional object of the invention is to allow end-users to place orders for perishable products with different time windows. A feature of the invention is that the end-user can specify both the item to be purchased and the time of delivery while placing the order. A benefit of the system is that the end-user has the flexibility to select both the time and place of delivery.

A further object of the invention is to provide a constant pickup location for a diverse variety of products. A feature of the system is that in one embodiment, a variety of suppliers provide products to a single distribution point. A benefit of the system is that a single distribution point can be used to pick up a large variety of products.

An additional object of the invention is to provide a system of distribution of perishable products that minimizes waste. A feature of the invention is that the system can indicate the expected demand for products based on real-time orders and expected patterns of demand. A benefit of the invention is that perishable items are not supplied to distribution points only to be wasted.

A further object of the invention is to gather information about products being offered by suppliers. A feature of the invention is that end customers can include ratings and otherwise indicate the level of satisfaction with a purchase. A benefit of the invention is that suppliers of products can see how pleased potential customers are with new offerings.

Another object of the invention is to provide real-time updates about the status of each distribution point. A feature of the invention is that the system includes a variety of sensors at each distribution point. A benefit of the invention is that the operator of the system can maintain product quality consistently throughout the chain of distribution.

A further object of the invention is to provide a system of distributing food with limited personal contact. A feature of the invention is that end customers interact with a meal distribution machine rather than a clerk. A benefit of the invention is that consumers can retrieve healthy and fresh food without interacting with another person.

An additional object of the invention is to sanitize items when food is delivered. A feature of the invention is that, in some embodiments, the distribution point is equipped with UV-emitting lights. A benefit of the invention is that food containers and other items can be sanitized before they are distributed to the end consumer.

A system and method of product distribution are described. At least one physical distribution point is used. End-users of the system place orders and designate a time and location of product pickup at a distribution point. The inventory at the distribution point is replenished based on demand expressed by the end-user orders.

### BRIEF DESCRIPTION OF DRAWINGS

The invention, together with the above and other objects and advantages, will be best understood from the following detailed description of the preferred embodiment of the invention shown in the accompanying drawings, wherein:

FIG. 1 depicts a schematic overview of one embodiment of the invented system.

FIG. 2 depicts an overview of a sample interface screen.

FIGS. 3A and 3B depict additional sample interface screens.

FIG. 4 depicts a flow chart of the operation of one embodiment of the system.

FIG. 5 depicts a flow chart of the installation and use of one embodiment of the system.

FIG. 6 depicts another view of an embodiment of the system.

FIG. 7 depicts a flow chart of an embodiment of a container return module.

FIG. 8 depicts a schematic overview of one multi-module deployment.

FIG. 9 depicts a flow chart of an embodiment of a drink dispenser, per one embodiment.

FIG. 10 depicts a flow chart of one embodiment of an interconnected refrigerator.

FIG. 11 depicts a flow chart of one embodiment of an interconnected module deployment.

### DETAILED DESCRIPTION OF THE INVENTION

The foregoing summary, as well as the following detailed description of certain embodiments of the present invention, will be better understood when read in conjunction with the appended drawings.

To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random-access memory, hard disk or the like). Similarly, the programs may be standalone programs, may be incorporated as subroutines in an operating system, may be functions in an installed software package, and the like. It should be understood that the various embodiments are not limited to the arrangements and instrumentality shown in the drawings.

As used herein, an element or step recited in the singular and preceded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

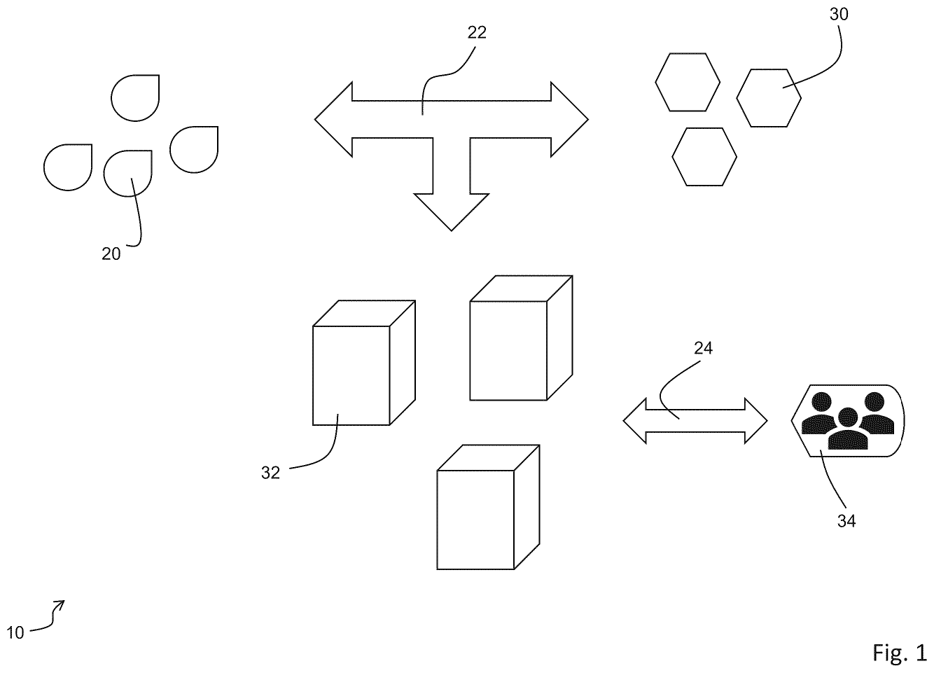

Turning to the figures, FIG. 1 depicts an embodiment 10 of the invented distribution system. The primary components of the embodiment 10 include the source producers 20, the manufacturers 30, the distribution points 32, and the customer interface 34.

The source producers 20, manufacturers 30, and distribution points 32 communicate to exchange information using the information exchange system 22 depicted as multiple arrows. While the information exchange 22 is depicted as arrows, the system is more than a passive means of exchanging information in one embodiment. The information exchange 22 comprises a means to model demand for products, communicate with the participants in the system, such as manufacturers 30 and producers 20. The information exchange 22 system is also responsible for communications 24 between the customer interface 34 and the information exchange 22.

In instances where the system 10 is being used to distribute perishable foods, the source producer 20 would refer to the sources of the ingredients, such as the importer or the farm responsible for the food item. The manufacturer 30 would be the restaurant or the catering company that turned the food provided by the source 20 into a sellable item. In some embodiments, the producers 20 and manufacturers 30 are combined into a single entity. For example, a farm may include an on-site food processing plant, such as a dairy containing a cheese-making facility. The distribution points 32 are temperature-controlled, secure vending devices that are filled by the manufacturers 30 or producers 20. At the time the vending devices are filled, the information exchange 22 system is notified so that an appropriate notification is triggered on the end-user interface 34.

While in some embodiments, the distribution points 32 include temperature control features, in other embodiments, where the products being sold are shelf-stable, the distribution points 32 do not use temperature control.

Each distribution point 32 communicates with the information exchange 22 to determine which items will be picked up at the respective distribution point 32. End users establish accounts in the information exchange 22 to place orders.

In the embodiment shown in FIG. 1, the distribution points 32 can only be accessed using the end-user interface 34 and do not operate as traditional vending machines that allow end-users to place orders by interacting with the vending machine directly.

The distribution points, in one embodiment, are devices with both cooled and heated sections. This allows each distribution point to serve both hot and cold items. The distribution points 32 are designed to minimize the amount of time that each end user requires to pick up their order.

The overall purpose of the system 10 is to improve the quality of convenience food available to the end-users. Worldwide, there are billions of workers in workplaces such as factories and offices. The system 10 provides a regular way to access fresh food of high quality. The amount of time each item has spent waiting is known, and the conditions under which the item was waiting are also subject to strict control in one embodiment.

The manufacturers 30 and the producers 20 benefit from the use of the system 10. These entities have access to new markets, for example, within a closed campus of an institution. Since the distribution points 32 are designed to provide highly perishable foods, the producers 20 and manufacturers 30 will need to be from the local area.

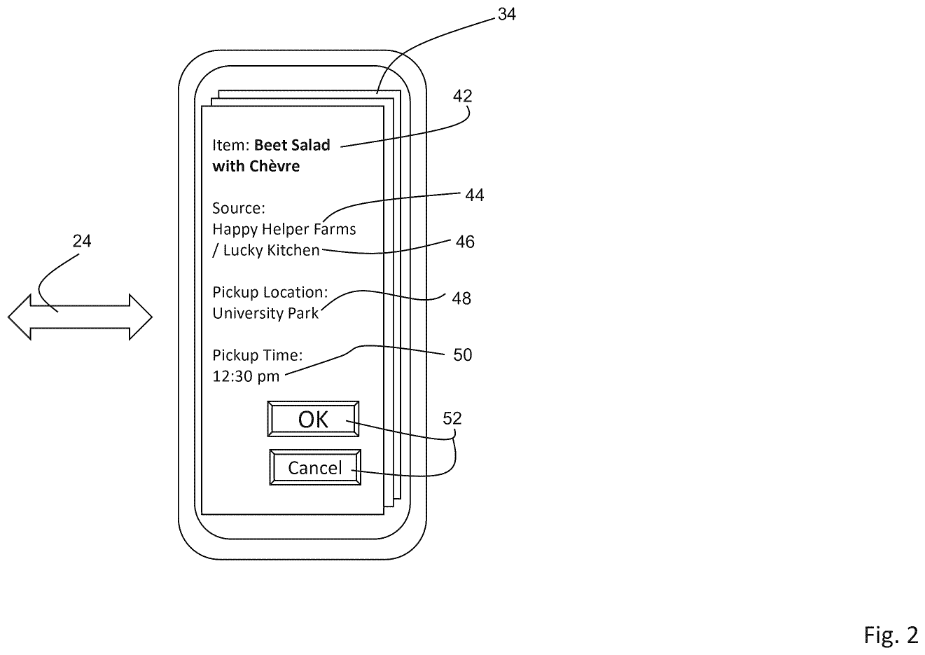

The customer interface 34 comprises a means for each end customer to request inventory from the system 10. A sample end-user interface is shown in FIG. 2. Each end-user has access to a personal account where the end-user can provide information about preferences, dietary restrictions, and any allergies. At the time of pickup, the end-user can retrieve their order at the desired time, at the requested place, with a guarantee that the order will be exactly as requested. Unlike at a conventional dining establishment, the pickup process can take a few seconds without requiring the end-user to travel to a particular restaurant. The same distribution point 32 can provide one type of meal on the first day and an entirely different meal on subsequent days.

The distribution of perishable inventory using the system 10 will limit the amount of wasted food and decrease the amount of energy and costs required for the logistics of multi-point distribution. Each supplier 20 and manufacturer 30 can reach a particular market by filling the distribution points 32 and without having to establish a physical presence at a particular location.

In one embodiment, the information contained within the information exchange system 22 is distributed to multiple end-users by using a blockchain-based record-keeping system. The resulting system 10 has a record of delivery from local suppliers, and each supplier is a confirmed source within the system. In one embodiment of the system, the implementation of blockchain technology allows end-users to review multiple levels of the supply chain, including the manufacturer or distributor. In such an embodiment, for a selected meal, the end customer will know not only the final producer, but the origin of the ingredients used to complete the order.

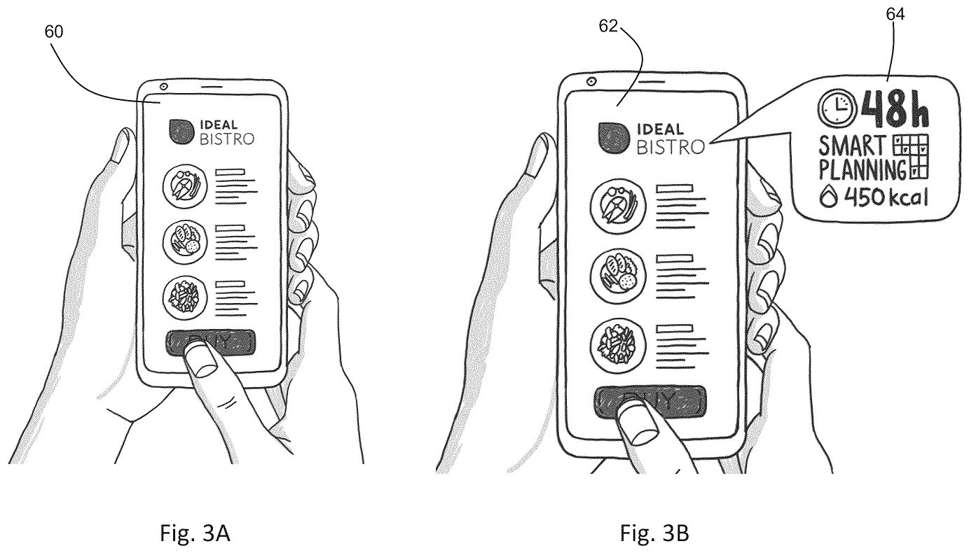

Additional sample interface screens 60, 62 are shown in FIG. 3A and FIG. 3B. As shown in FIG. 3B, the interface screen includes an indication of the total nutritional value of foods 64 consumed during a specified time interval.

As shown in FIGS. 3A and 3B, the user interface is implemented by an app running on an end user's multi-purpose computing device, such as a tablet, smartphone, laptop, or other computing device. The multi-purpose computer is connected directly or indirectly to a central computer system, using a wired or wireless connection such as a local network at a distribution point or a Wi-Fi connection to the internet. The multi-purpose computer device comprises memory, one or more central processing units, a user interface display, and a user interface device such as a touchscreen. The multi-purpose computing device is programmed with computer-executable instructions that are stored in the computer memory and are executed by one or more central processing units, which perform operations to complete the steps described below.

The backend functions described herein are performed by at least one server, in one embodiment, or a flexible number of servers that provide a changing amount of computer system resources as demand increases. The server or servers provide sufficient data storage and computing power to fulfill the tasks outlined herein, with each server comprising at least memory, at least one processing unit, data storage, and computer-readable instructions.

### Data Analysis

The information exchange 22 system performs several types of data analysis to aid in the operation of the system 10. In one embodiment, the analysis comprises modeling the demand at a particular location and creating a profile for that location. While a variety of factors are used to model demand, in one embodiment, these include:
The character of the work being performed at the particular workplace as there are differences in demand between industries and types of employers.
The number of employees working at a particular location.
Whether the employees will receive additional reimbursement for purchases, such as incentives for buying a healthier lunch option.
The number of locations to which food must be delivered.
The number of shifts in use at the particular location and the working hours at the location.
The number and length of food breaks for employees, and the times that the employees generally use for eating.

In one embodiment, following analysis of these and other factors, the information exchange 22 system will suggest changes to the suppliers 20 and manufacturers 30.

The above factors are also used in initially selecting a location for the distribution point 32. The distribution point 32 is customized with individual accounts managed by the system 10.

### Sample Interface

A sample interface 34 is shown in FIG. 2. The end-user can select an item 42. For many such items 42, the source 44 and the producer 46 are also included. In this way, the end-user can select desired producers if they have a preference for a particular producer.

The end-user can select a particular pickup location 48, and a pickup time 50. Finally, the end-user can act on the order by either confirming it or canceling it. The interface 34 communicates with the information exchange system 22 using wireless communication means 24, shown in FIG. 1, and FIG. 2.

When picking up an order, the end-user interface 34 will display a unique identifier, such as a bar code or password, that the user provides a specific distribution point 32 to be able to pick up an order.

In summary, the system provides multiple benefits. For an employee, the system allows for the ordering of customized, fresh, high-quality meals that can be picked up at a convenient location. The employee can remain focused on other responsibilities. For an employer, the system provides a valuable workplace perk and allows the company to compete with other companies that offer a variety of eating options. The employer can be located in an office space that is further away from trendy eateries while providing employees access to fresh dining options on-site. For the foodservice operator, the system provides a consistent customer base and revenue flows that can be planned for. The system prevents the waste and costs associated with standalone dining establishments. The environment benefits by avoiding wasteful food production, thereby decreasing the use of chemicals and other stresses on the environment. Preordered food means a planned and thoughtful choice. Food production vs. needs score is 1:1. There is limited waste, less water use, and less pollution.

The system allows an employee to preorder preferred meals and to collect them at the office without a prolonged wait. The employer company can offer its customers or employees a built-in technological solution that outrivals other available solutions in nutrition. Customers benefit from easy, convenient access to healthy meals from a local caterer that are delivered directly to the workplace at a specific time.

In one embodiment, the system is referred to as a Meal as a Service, or MaaS.

In one embodiment, the goal is to improve the lives of billions of employees in factories and offices by providing them with regular access to fresh, high-quality meals produced by local suppliers. To achieve this goal, in one embodiment, referred to as the Ideal Bistro system, allows anyone to plan their own diet and order their favorite meals even one month in advance. Each employee is able to pick up the meal at a convenient time of the day at a location as close to their workplace as possible with a 100% guarantee that they will get what the employee chose. In one embodiment, the whole process will take 3 to 4 seconds. The system significantly reduces the amount of food waste.

### System Deployment

In one embodiment, the deployment of the system consists of the following steps.

First, food producers are chosen according to criteria agreed upon by the workplace, which include expectations of the number, type, and nutritional value of the meals on the menu. Prior to selection, each producer is verified in the way the meals are prepared and transported in order to maintain their quality and freshness. Further, potential partners are verified for compliance with legal requirements. Only local companies that are able to meet the expectations are chosen as potential suppliers or manufacturers.

In one embodiment, at the same time, the customer's requirements related to the need to provide meals to the employees of their company are quantified and analyzed. When working out the system assumptions, the system takes into account the specific working conditions of our customers, especially:
a. The character of the work being performed at the particular workplace, as there are differences in demand between industries and types of employers.
b. The number of employees working at a particular location.
c. Whether the employees will receive additional reimbursement for purchases, such as incentives for buying a healthier lunch option.
d. The number of locations to which food must be delivered.
e. The work shifts in use at the particular location, and the working hours at the location.
f. The number and length of food breaks for employees, and the times that the employees generally use for eating

On the basis of information resulting from the analysis of a combination of many factors, the offered system is customized. Importantly, the system as implemented is custom for a particular deployment and addresses the gap resulting from the limitations of the existing solutions.

In the last stage, on the basis of a previously prepared plan, the implementation of Ideal Bistro is initialized. The system consists of:
a. The application (Pay2vend)—(AI, Big Data), a system of individual consumer accounts that manages the whole process of ordering and receiving meals in Ideal Bistro.
b. The distribution points or “foodomats” and other machines (IoT), managed by a service, such as one provided by Pay2vend, which allow anyone to freely receive individual orders within 3-4 seconds.

The deployment and implementation occur in two tracks, in some embodiments:
a. First, user accounts are created in the application, which allow users to:
place orders—in the user panel, we place a menu with all necessary information, such as weight, nutritional value, and allergy information
pay for an order—the user manages his/her payments
view order receipts
b. Then, in indicated locations, Foodomats and other machines are used to collect meals and food.

The end result is a technology that supports companies/schools, and other locations in improving the health, productivity, and job satisfaction of their employees.

### Impact on Existing Distribution Methods

In one embodiment, the system is designed to influence the environment from the distribution of goods to the distribution of value. Looking for an answer to the question of how to better provide food at work, the system provides a solution that will resolve several issues:
1. The system enables billions of employees worldwide to take care of their own well-being and health at the peak of their activity during the day.
2. The system makes it easier for millions of businesses to take care of their employees by organizing a convenient process for accessing personalized food at work that can also be used to take food home. A company can highlight the value of employee health by financing their meals.
3. The system reduces the amount of wasted food by planning it at every stage of the supply chain.

The system is designed to support efforts at fighting against the diseases of the 21st century, i.e., obesity or diabetes, supporting preventative actions. The effects of these actions have a measurable impact on the economy. Another opportunity presented by the system is the collected data on the daily shopping habits of hundreds of millions of consumers to improve the distribution and use of food worldwide. The system facilitates the production of food in millions of small, local kitchens, creating a new ecosystem. New products are developed and manufactured on the assumption that they will be consumed within a short period of time. Even candy bars, snacks, and drinks can be fresh and healthy. Thanks to technology, the consumer will be able to verify where the ingredients and raw materials of the dish come from. In one embodiment, hundreds of millions of distribution points change the way meals are distributed and allow customers to receive them conveniently in the workplace.

The system competes with entities such as mass catering in companies. At the moment, they are mainly internal cafeterias and catering companies. These solutions generate problems both for the company and for the suppliers of the service themselves. Based on the organizational aspects, there are five major limitations of the traditional model:
1. The distances between the cafeterias and the individual production halls are large enough for a part of the crew to be unable to take advantage of them due to the excessive time needed to reach their destination
2. Serving simultaneously, for example, several hundred dishes takes too much time, and a part of the crew resigns from a meal at the sight of the line of waiting people
3. The process of meal serving/purchasing itself takes more than 2 minutes per employee because the consumer usually makes his or her choice at the time of purchase, plus the time it takes to serve and pay
4. The cafeterias operating in the factories typically do not operate 24/7
5. Dishes sold in the cafeterias are usually very limited in variety, which makes it impossible to respond to the individual nutritional needs of employees.

In one embodiment, the system eliminates the limitations resulting from the hitherto existing model of organization. The embodiment's approach, in this case, goes beyond the prior art. The system is not a food producer, and other companies involved in this business are valuable partners. Thanks to the distribution method, an embodiment creates a model of food distribution from scratch in the company, but it can also provide a valuable supplement to the existing traditional solution. When proposing a completely new solution in a company with an organized food distribution for employees, the system does not need to eliminate the old, well-known, safe system and introduce a new, unknown solution.

In summary, one embodiment of the system is aimed at people who want to improve their quality of life through better nutrition. There are many people declaring that they are working towards this goal. However, most of them never achieve their goals, mostly because of the hardships of everyday life. In one embodiment, the system allows a user to reach their goals with little effort. Once in a while (even once a month), each of them has to plan what they would like to eat in the near future. Planning gives measurable benefits to each of the participants in the process, which is also the key to achieving another important social goal, which is to reduce the amount of food waste. In one embodiment, the system works by:
1. The consumer orders in advance; in some embodiments, the consumer can order within 30 minutes of pickup if selecting from a fixed menu.
2. The company preparing meals has to collect the required ingredients in the necessary quantities and prepare for the production process (nutritionally valuable dishes usually require more time to prepare).
3. An important element of the process is the fact that while accepting an order in a shopping cart, the customer simultaneously pays for it—it gives the supplier 100% confidence that each order will be paid for, which has an impact on the reduction of the price of the meal.
4. At the end of the day, the consumer gets a meal that meets his or her expectations at an attractive price, sure to be available for pick up at a convenient time
5. A catering company can accurately plan its purchases, minimizing food waste from unused and unsold meals and ingredients.

In one embodiment, the system has the potential to create a new global marketplace and an innovative food distribution process. The embodiments use workplaces as locations for the collection of food. The embodiments engage in new technologies in the MaaS (Meal as a Service) process.

In one embodiment, the system's deployment requires the design and implementation of the process of mass nutrition in companies that are concerned with the well-being and health of employees. The deployment adapts to the specific conditions of each of the companies, so while designing each solution, the installation takes into account many factors resulting from the specificity of work of a company to the geographic location, and the availability of producers. The compatibility of Ideal Bistro with existing solutions on the market gives the system a wide range of possibilities. In a selected company, an installation creates a dedicated solution from scratch or adapts to the existing ones, filling in the gaps resulting from the limitations of the existing food system. A good example of this is cafeterias, which are usually not able to serve companies working in 24-hour mode. Identifying specific deficiencies, we are able to quickly correct them on the basis of foodomats (IoT), i.e., devices that allow you to collect ordered meals within four seconds, and pay2vend.com technology (AI, Big Data, Blockchain).

The development of technologies related to Ideal Bistro allows the system to achieve its main goal. The system creates a platform for the exchange of information between food producers, suppliers, and consumers (Industry 4.0). The system is used to reprogram the distribution process. The system acts as a tool that makes it possible to change a typical food consumer into a prosumer.

In one embodiment, Ideal Bistro is not limited to providing food for company employees. The system addresses the challenges faced by employees during a working day when they are tempted to make the wrong dietary decisions. We believe that thanks to us, proper nutrition will supplant actions aimed at eradicating hunger. The system creates new expectations and rules for eating by providing MaaS-Meal as a Service.

In one embodiment, the system allows the end customers to choose a food product on many levels: the origin of the ingredients, the date of production of the ingredients, the manufacturer, the date of production of the food, and many others that are important to the end customer individually. The system, in one embodiment, creates a reality where food is produced in millions of small local businesses without needing contact with each individual end customer. The technology gives direct business interaction between end-users and food suppliers at every stage, without requiring in-person interaction. In one embodiment, the system engages managers and business decision-makers to co-create the new food distribution network that can create the places where we spend most of our time in our adult lives-offices and factories. The system supports taking food home from there.

### Summary of Use of One Embodiment

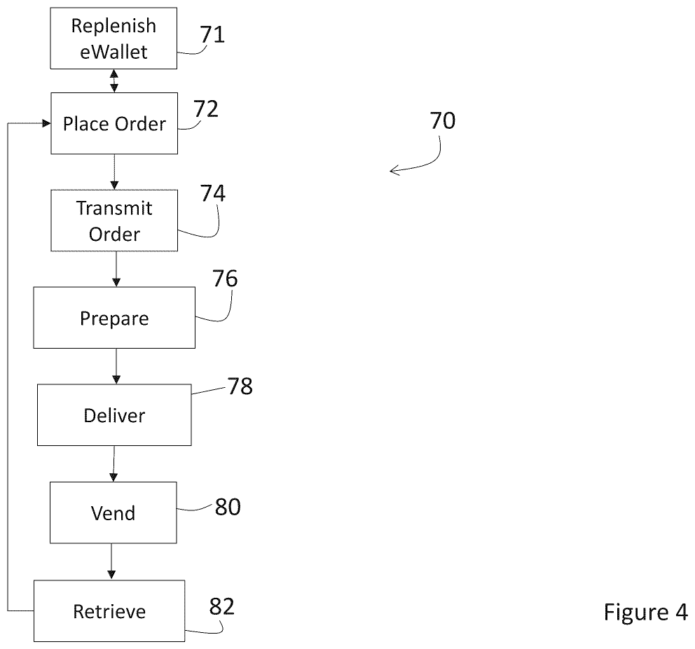

Turning to FIG. 4, depicted therein is a flow chart showing the use of the system pursuant to one embodiment 70. The process begins by the end-user placing an order 72 and paying for their order from an electronic wallet. If needed, the end-user will replenish their electronic wallet 71 prior to placing the order, in some embodiments. In some embodiments, all orders are prepaid at the time of pick up from an electronic wallet balance. In this way, the end-user does not need to pay for the order at the time of pickup. Funds are already available, and the pickup can proceed as quickly as possible.

As detailed above, placing the order 72 within the system allows the user to select not just fixed menu items, but in one embodiment, the user interface allows the customer to select details such as suppliers and types of ingredients. In one embodiment, the end customer can select that certain elements of the food item be organic, such as root vegetables, and others can be grown conventionally.

In one embodiment, the order placement 72 occurs with at least a 48-hour lead time. In other embodiments, the order placement 72 occurs with a variable lead time, depending on the type of item being ordered. For example, ready-to-eat items may not require any lead time.

Once the end customer places the order 72, the system transmits the order 74 to all participants in the supply chain. The system designates one participant as responsible for delivery of the finished product; in one embodiment, for example, a restaurant may be the designated responsible party for delivering the ordered item.

Upon receiving the transmitted order 74, the supply chain participants will prepare 76 the order. During the preparation step 76, in one embodiment, the system allows the end customer to see the progress of the order. In another embodiment, the system allows the end customer to make changes to their order during preparation, by, for example, requesting additional ingredients or making substitutions.

Following preparation and packaging, the responsible party will deliver 78 the order to the distribution point. During the delivery step 78, the system ensures the freshness of the order by tracking temperature ranges and any delivery delays. Suppliers that fall below quality metrics, for example, by taking too long to deliver items, are noted by the system. In one embodiment, the information about performance by each supplier is shown to the end-user as part of the ordering interface so that the customer can decide whether to place the order using that particular supplier.

Following delivery, the order is placed in the distribution point and vended 80 to the end customer. The distribution point will complete the finishing steps, such as reheating the food. In one embodiment, the distribution point includes a sanitizing system, such as one using ultra-violet light, sanitizing solution, or a combination of several sanitizing methods. The exterior of the order container is therefore sanitized, in one embodiment. In another embodiment, the distribution point provides reusable utensils to prevent creating waste, and the reusable utensils are sanitized before being given to the end-user.

As discussed above, in one embodiment, the end customer can select a time when the order is to be retrieved 82. At the time the order is due to be retrieved, the distribution point will alert the end customer by sending them a notification or another message through the app interface, as shown in FIGS. 3A and 3B.

At the time the end-user is retrieving 82 the order, the end-user is provided additional information, such as information about the chain of supply, in one embodiment. The distribution point, therefore, acts as an additional means of advertising in one embodiment. The distribution point can also provide non-commercial messages, such as health information or reminders to the end customer.

The embodiment of the system as described above uses the following features to accomplish its goals in a novel way: a U.V. disinfecting lamp at the distribution point or vending machine, a mobile application which is used to retrieve orders from compartments, a backend database which maintains a chain of responsibility for the order starting with the initial supplier (such as a farmer). In one embodiment, the system also supports a virtual wallet to facilitate payments.

In at least one embodiment, the system includes communications between various devices, such as a coffee machine or a dispenser of fresh fruit juice or another drink dispenser.

In one embodiment, the system includes an artificial intelligence-driven suggestion algorithm that will provide suggestions for orders to customers. In at least one embodiment, the suggestion algorithm performs automatic orders on behalf of customers.

In one embodiment, the distribution points are stationary and found in defined locations, with the client choosing which location to pick up the order and the time of pickup. The orders are prepared by certified providers, such as restaurants, and are not simply reheated foods typical of vending machines. The range of foods available at the applicant's system is much higher than the variety of foods that would be available if the food had to be prepared locally. For example, such robotic food preparation vending points are limited to offering foods such as pizzas and crepes.

The present system focuses on individual orders and the customer experience at the time of pickup, and not on efficiency in the distribution chain. Many solutions exist to improve the logistics of distributing large quantities of groceries, including using vending machines. In such systems, there is no opportunity to customize the order, as is possible with the present system.

In the embodiments described above, the distribution points do not prepare meals. The distribution points facilitate the pickup of a large quantity of custom orders in a short amount of time. The orders were placed previously and by registered users who pre-paid using a virtual wallet. The process is subject to supervision and control, including prevention of contamination and by limiting the need to interact with staff at the time of pickup of the food order.

As described above, the system provides a meal as a service (MaaS) to individuals and employers. On the employer side, the system empowers employers to provide workers with fresh, healthy meals by making the process of food delivery safe and secure. The foods offered by the organization are high-quality and delicious, without forcing the employees to leave the work area. The primary components are a smartphone app and a distribution point referred to as a foodomat in one embodiment.

The system solves the problem faced by many employers who want to provide a safe work environment without employees congregating at lunchtime. The system allows employees to retrieve healthy and fresh foods from a safe and hygienic location without interacting with another person.

In use, the system allows employees to place orders and pick up meals. Over time, the machine learning and AI algorithm, the system will provide suggestions both to consumers and to participants in the distribution channel to improve preparation logistics of the meals by predicting meal ordering patterns and optimizing nutrition plans for each user.

As part of one embodiment, the system provides a process and technological solution that interconnects foodomats with other devices that prepare foods or drinks. For example, in one embodiment, the foodomats are connected in a system with a juicer that extracts fresh juice from oranges, apples, and other fruits. In another system, a foodomat is connected to a coffee maker that grinds fresh coffee and dispenses fresh cups of coffee. All processes related to the selection of a main entre or product along with the drink and the payments occur within a single centralized application for these ancillary devices as well as the foodomats. The process of pickup of the food or drink involves placing the phone or other identifying card or token, which identifies the order and the user. At that time, the previously selected and paid-for product is dispensed to the user. In this embodiment, the main food item is dispensed from the foodomat while concurrently, the drink is dispensed from the drink maker (such as fresh juice from the juicer).

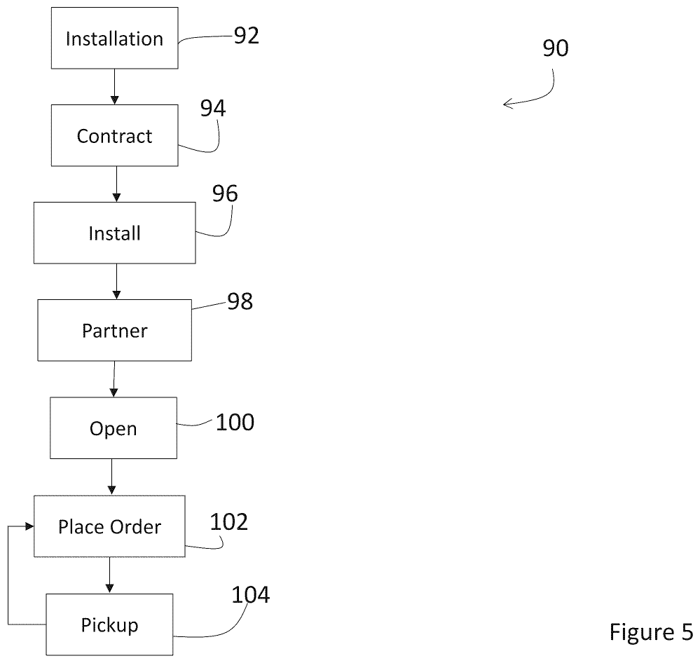

As shown in FIG. 5, the system rollout 90 occurs by first installing 92 a distribution point or foodomat. The party responsible for the location will sign a contract 94 and install 96 the device based on the customer's expectations of use. A food specialist then contacts the responsible party to partner 98 with local restaurants and plan a menu to meet the need of the location.

Once installed, the foodomat is made available 100 to consumers. Each user selects a meal along with the day and time of pickup 102 from the associated app. The orders are then prepared and delivered to the selected foodomat. The consumers then pick up order 104 after being notified of the delivery.

The pickup occurs in a safe and convenient manner from the designated foodomat. The customers do not have to leave the premises. In one embodiment, the foodomat doors are opened using scanning of a contactless means, such as scanning a barcode or reading the code from an NFC chip. In such embodiments, the consumers do not have to touch any control panel on the foodomat. Their order is presented to them after the consumer presents the appropriate order retrieval token.

A benefit of the system is that it allows for the building of a highly hygienic and maintenance-free cafeteria, which also provides a large variety of food items.

In order to finance the system and especially the distribution point, in one embodiment, the customers pay a monthly subscription fee, which can be offset by the employer and offered as a benefit. The food ordered through the system will include a margin that is shared with the host of the distribution point.

In one embodiment, the system includes a specially designed payment system. In an embodiment, the payment system comprises a virtual wallet where the end-users transfer funds to the virtual wallet. The orders are pre-paid, allowing for fast pickup. If an end-user is dissatisfied with their order, they can submit a refund request while interacting with the system app. The end-user does not need to contact the operator of the distribution point or the supplier of the order. The processing of the refund is done within the app. Information about refunds is transmitted to the system and the recommendation engine, in some embodiments. In such embodiments, the end-user will not receive recommendations to order from suppliers who have similar offerings as suppliers where the end-user has previously requested refunds. In one embodiment, the system allows the end-user to specify the reason for requesting a refund. For example, the end-user may have been unhappy with the type of food or the container it came in, or the freshness of the food. In one embodiment, the system will also disfavor suppliers with multiple refund requests.

In some embodiments, the end-user can add a tip to their order either at the time the order is placed, at the time of pickup, or after the order has been consumed. The system app facilitates the tip function, which is likewise paid for using the end user's electronic wallet, in one embodiment.

### Additional Embodiments

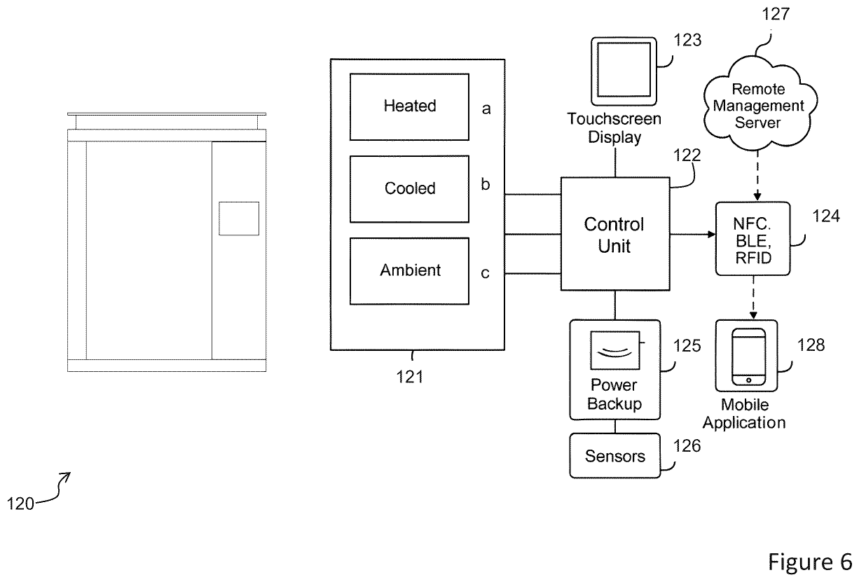

In one embodiment, shown in FIG. 6, the system comprises a food dispensing unit (a foodomat) with at least one temperature-controlled locker and wireless access.

Existing food lockers and vending machines typically lack flexibility in temperature control and offer limited user authentication methods. Furthermore, few solutions allow for simultaneous access by multiple users or remote, cloud-based management with real-time data synchronization. There exists a need for an integrated system that allows precise temperature regulation, secure wireless access, and seamless remote administration.

The embodiment shown in FIG. 6 provides an automated dispensing unit comprising a plurality of lockers, each configured to maintain a specific temperature range, for example, heated, cooled, frozen, or ambient. The system includes a local control unit that manages locker access, sensor monitoring, and user interactions via an integrated touchscreen. Authentication is provided via user-entered codes, contactless cards, smartphones, or smartwatches using NFC, BLE, or RFID. The system is managed via a remote platform, and locker access can be synchronized with a mobile application. A battery backup ensures operation in the event of a power failure.

The embodiment shown in FIG. 6 depicts an intelligent, modular food dispensing unit (foodomat) comprising multiple secure storage lockers configured for heated, cooled, or ambient storage of meals and perishable items. The unit includes a touchscreen interface, wireless user recognition systems (NFC/Bluetooth/RFID), internal sensors for temperature and environmental monitoring, and an integrated local control unit. The device may be accessed via a mobile application or contactless media and is capable of remote management through a cloud-based platform. The system supports simultaneous or near-simultaneous access by multiple users and includes a battery-powered backup module for uninterrupted operation.

The embodiment 120 consists of a set of lockers 121. Each locker 121 is thermally insulated and equipped with a heating or cooling module, such as a thermoelectric Peltier device, a convection heater with fan, and other heat exchange mechanisms. A digital temperature sensor monitors the internal environment, and each compartment can be individually set to a target temperature.

The system also includes a touchscreen display on the front panel in communication with the lockers 121. The front panel includes a touchscreen interface for retrieval code entry, displaying order information, user communication, and status and alert messages.

The system includes a wireless communications 124 module. The wireless communications 124 module provides wireless access and authentication, along with other services. The wireless communications 124 module, in one system, supports: NFC, for smartphone and card-based access, Bluetooth Low Energy (BLE), for mobile app interaction, RFID, for quick user identification, One-time access codes, displayed or entered on-screen, including, for example, by the display of a QR code. Multiple users may open lockers concurrently within a one to five second window.

A Local Control Unit is also part of the embodiment 120. The local control unit includes a processing unit that manages: locker access mechanisms, Environmental sensor readings, user interface logic, and communication with cloud servers.

The sensor system 126 includes multiple modules and sensors. It includes temperature sensors in each locker, zero or more humidity sensors, and optional proximity/motion detectors.

In some embodiments, the system includes a power backup 125. The rechargeable battery module ensures continued locker access, data protection, emergency temperature maintenance (limited time).

The system also includes a remote Management Platform. The foodomat connects to a remote server via Wi-Fi, LTE, or Ethernet, enabling live monitoring of status and usage data, remote firmware updates, alert notifications, and integration with third-party systems.

As shown in the embodiment, a mobile app integration is also offered. The mobile application provides: meal order/reservation and notification features, locker unlocking via QR/Bluetooth/NFC, user identity verification, and order tracking.

FIG. 6 depicts a block diagram of the system providing an overview of the architecture. Multiple temperature-controlled lockers (a, b, c) within the set of lockers 121. Also shown in the control unit 122, the display/interface module 123, the wireless authentication system 124, and the power backup module 125. Finally shown are the temperature sensors 126, the remote management server 127, and the Mobile application interface 128.

### Return and Reuse

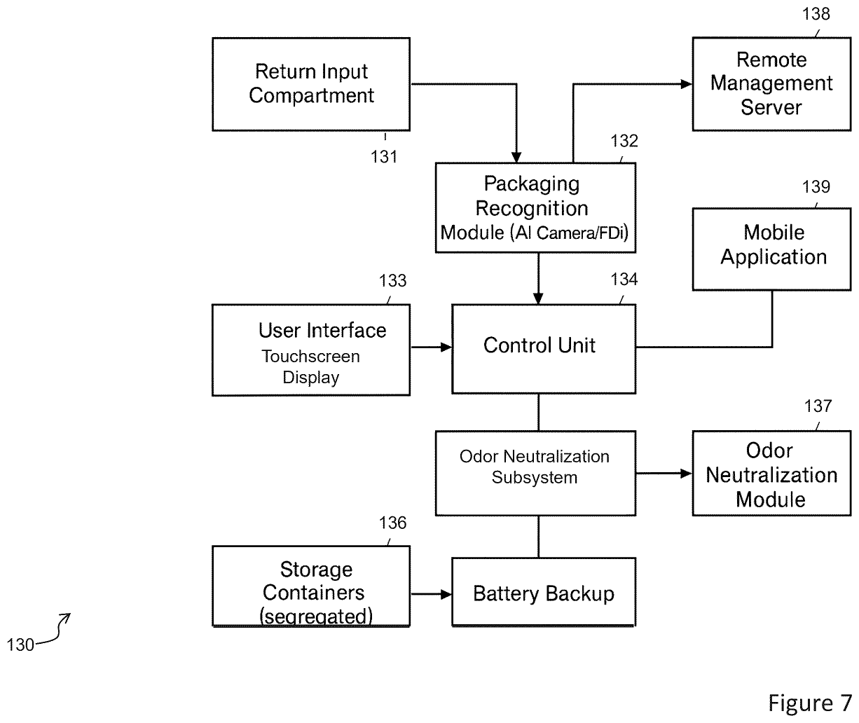

In one embodiment, shown in FIG. 7, the distribution point further includes an integrated return module 130 configured to accept used packaging and serving utensils from the end-user.

In one embodiment, this module includes individual compartments for utensils and packaging, with optional sub-sorting for recyclable and compostable materials. In a preferred embodiment, the return module includes a sanitizing subsystem, such as a UV-C light chamber or chemical cleaning bath, to pre-treat returned items to reduce contamination and odor before transport back to a processing facility.

Returned items are scanned or tagged in one embodiment, allowing the system to associate returned utensils with specific users for incentive or deposit return programs. This promotes a circular use model where customers are encouraged to return reusable containers and utensils, enhancing environmental sustainability and reducing single-use waste.

The scanning of returned items also allows for crediting of the end user for collecting and returning packaging and serving trays.

The data exchange system tracks returned items and integrates this data into user profiles, enabling feedback, accountability, or loyalty-based reward systems. In some embodiments, the return module is designed to operate touchlessly and utilizes contactless scanning to authenticate and log the return of items.

In at least one embodiment, the return module operates contactlessly, using sensors to detect item returns and authenticate the user without requiring physical interaction.

In one embodiment, the return module includes a camera that detects the item to be returned and will highlight whether the item for return should be discarded or placed in the return module. In one embodiment, the return module includes multiple apertures and an indicator, such as a light, that is illuminated to help the end user properly return the particular item.

The details of the embodiment of the return unit are shown in the schematic of FIG. 7.

An automated return station (ReTurno) designed to intelligently identify, accept, and store returned food packaging—both reusable and disposable—in hygienic and odor-neutral conditions. The system integrates user and packaging recognition via camera-based AI, RFID, or other identification technologies. It supports refundable deposit tracking and reward mechanisms and includes odor-neutralization features. ReTurno can be used as a standalone unit or stylistically integrated with an automated food dispensing unit (Foodomat). A battery-powered version is available for flexible deployment.

The embodiment 130 (ReTurno) relates to automated systems for handling food packaging returns, particularly those enabling identification, secure storage, and environmental control for reusable or compostable containers.

Single-use food packaging contributes significantly to global plastic waste. While reusable and compostable alternatives exist, there is no widely adopted, automated system for efficiently returning and managing these containers. Manual systems are inconvenient, inconsistent, and lack identification capabilities. ReTurno 130 addresses this gap by enabling secure, intelligent, and hygienic packaging return with traceability and incentive logic.

As shown in FIG. 7, ReTurno 130 is a smart return station equipped with the following modules. Packaging recognition systems using computer vision and/or RFID/NFC, User identification capabilities, Return validation with deposit or reward registration, odor-neutralizing internal environment, Secure internal storage with segregation options, Integrated touchscreen interface, connectivity to mobile apps and remote management servers, A stylistic and functional design matching the Foodomat unit.

The system includes a return input chamber 131. ReTurno includes one or more input modules through which users insert packaging. These are equipped with: Auto-sensing doors, internal compartments for storage and sorting, and optional self-cleaning baskets or removable bins.

Next, the system includes a packaging Identification System, which uses AI-driven cameras to recognize packaging models, dimensions, and conditions, RFID/NFC tags embedded in reusable containers, Barcode scanners for coded returns, and optional load sensors to confirm physical properties. Each item is logged with time, user ID, and container type.

The embodiment 130 shown in FIG. 7 includes a user interface or touchscreen display 133 providing functionality such as user authentication. ReTurno supports multiple methods: touchscreen input (codes, PINs), NFC or BLE from smartphones or smartwatches, Loyalty cards, access tokens, or other contactless devices, integration with a mobile app 139 for seamless user flow.

The system 130 provides incentives and refunds. The system enables: issuing a deposit refund for eligible reusable containers, reward point registration for returning compostable packaging, centralized tracking of container circulation, and integration with external payment or loyalty platforms.

The system includes an odor Neutralization Module 137. Returned containers 136 are stored in controlled conditions using: ozone sterilization, refrigeration or active cooling, air filtration with activated carbon or chemical neutralizers.

Sensors may trigger odor control automatically based on VOC detection.

The built-in display, identical or compatible with the Foodomat interface, guides the user through: identification and instructions, feedback on container recognition, and status updates on return or deposit handling.

It may be integrated physically and logically with the Foodomat unit to form one user flow.

The embedded control unit 134 subsystem coordinates all input/output devices, recognition and authentication logic, reward/deposit registration, and connectivity with cloud platforms or on-premises management systems.

The Remote Access and Monitoring panel allows: live monitoring of status, storage levels, and technical faults, remote diagnostics and firmware updates, integration into ESG, zero-waste, and CSR reporting platforms.

The system communicates via Wi-Fi, LTE, or Ethernet and includes Mobile Application Integration. Users may use the companion mobile app to receive deposit refunds, monitor return history and earned points, view eligible items and return rules, and interact with the unit via QR code or Bluetooth handshake.

The embodiment 130 includes Power Supply Options, including a rechargeable battery module. The module allows operation during power outages, installation in off-grid or semi-mobile locations, and compatibility with solar or hybrid energy sources.

ReTurno is adapted to be an ideal companion module for Foodomat, with seamless physical, functional, and visual integration. Units may be deployed together under a unified housing with consistent UI/UX, branding, and ESG compliance.

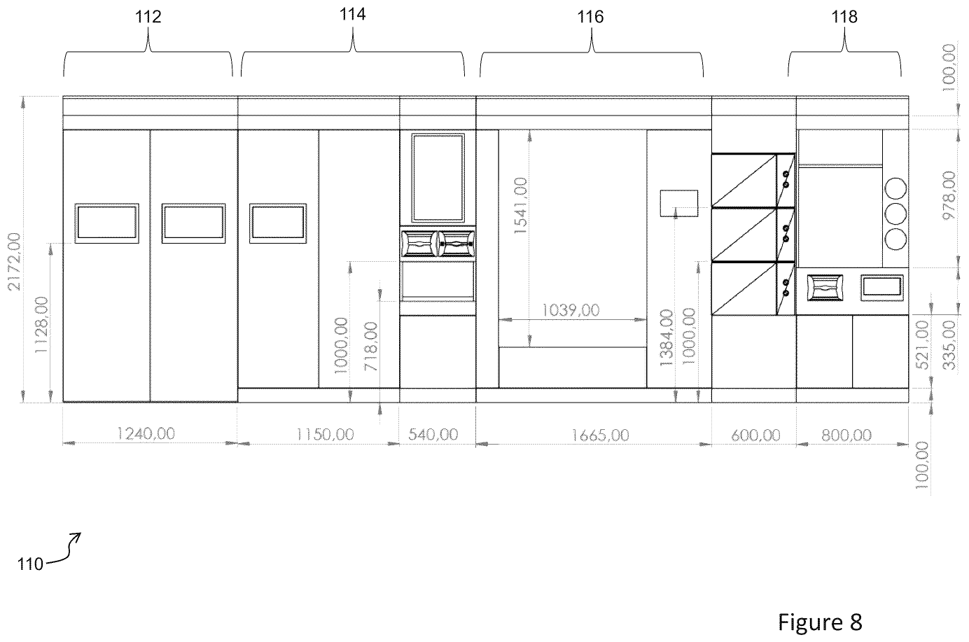

FIG. 8 shows several of the modules combined in one embodiment 110. Depicted in FIG. 8 is the return module 112, a set of accessories and trash bin 114, the food dispenser or foodomat 116, and the coffee and other drink dispenser module 118. While FIG. 8 shows several measurements, they are for reference only.

### Coffee and Drinks Dispenser

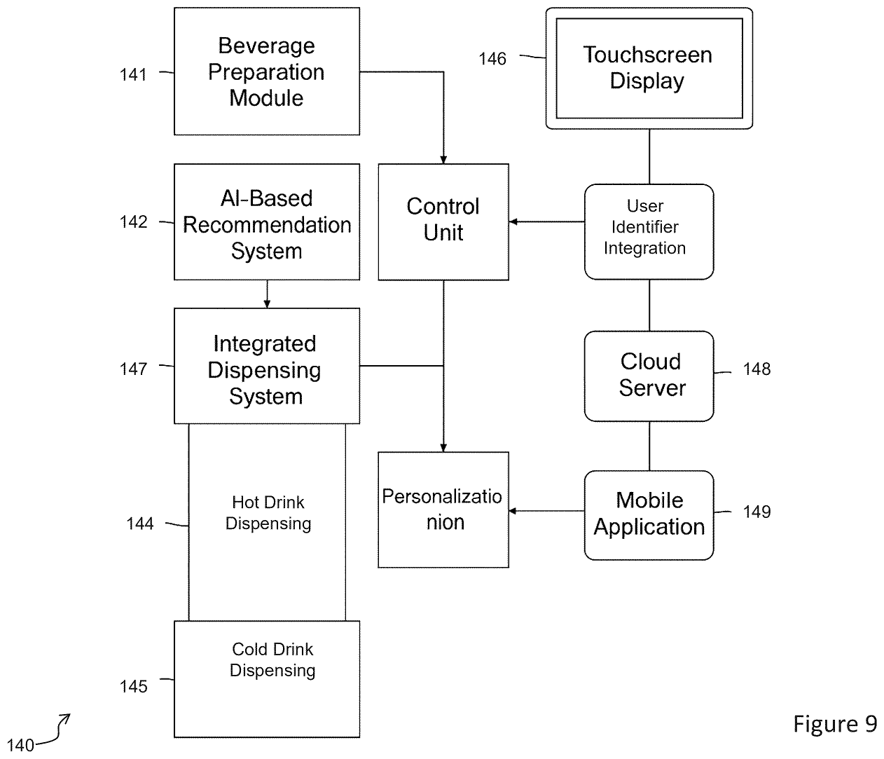

FIG. 9 shows a schematic overview of a coffee dispensing embodiment 140.

A modular 141, automated beverage dispensing unit capable of preparing and serving hot and cold drinks, including espresso-based beverages, instant and ground coffee, hot chocolate, milk-based drinks, juices, and water. The system supports recipes personalized through a mobile app or food distribution network (e.g., Foodomat), with integration of reusable or disposable cup return via ReTurno. The unit may dispense drinks with ice or flavored syrups and uses AI to suggest beverages aligned with user preferences and health goals.

The embodiment relates to automated beverage systems, particularly to smart dispensing devices for customizable hot and cold drinks, connected to a user preference database and integrated within a sustainable food service network.

Existing vending machines for beverages offer limited personalization and are typically not integrated with sustainability systems or digital personalization platforms. There is a growing need for intelligent, modular beverage systems that deliver health- or taste-optimized drinks in a personalized, reusable format with a returnable container lifecycle.

The embodiment 140 provides a smart drink dispensing system 147 that prepares a variety of hot and cold beverages, including espresso, instant and ground coffee, milk-based drinks, hot chocolate, water, juices, and ice drinks, allows for deep personalization of recipes via a user-linked app and backend system, and integrates with the Foodomat and ReTurno ecosystem. The embodiment 140 suggests beverages to users using AI based on taste preferences, time of day, and health indicators. It supports eco-friendly operation through compatibility with reusable cups and return systems, can be deployed standalone or in seamless physical integration with Foodomat and ReTurno.

The embodiment 140 unit includes: coffee modules for freshly ground espresso, instant coffee, ground or capsule-based blends. It includes milk modules for: natural refrigerated milk, powdered milk (dry mix reconstitution), dairy milk alternatives including almond, soy, and oat. It also includes a hot beverage module 144 for hot chocolate, tea infusion (optional), along with a cold beverage module 145 for water (still or sparkling), chilled juices, cold brew coffee, and ice dispensing.

The system includes flavor enhancement options, including flavored syrups (vanilla, caramel, etc.), sweeteners or plant-based additives, and functional powders (vitamins, protein, etc.).

In one embodiment, the system includes a personalization engine. Users can create and store custom beverage recipes, select preferences via mobile application 149 or touchscreen interface 146, link their profiles to the system via NFC, QR, BLE or cloud account. The device adjusts brewing parameters such as: strength, temperature, quantity, milk type, sweetness, and syrup addition.

The embodiment includes an AI-Based Recommendation System 142. The system uses machine learning algorithms to analyze user preferences, time-of-day, and context to suggest beverages (e.g., espresso in the morning, hydration-focused drinks in the afternoon), as well as health-related recommendations (e.g., reduced sugar, lactose-free, functional drinks) based on user input.

The system has an integrated Dispensing System. The output section includes: adjustable height platform for cups, sensors for cup presence, reusable or disposable cup compatibility, and an optional sterilization or pre-rinse system for personal containers.

The coffee system can be integrated with Foodomat & ReTurno. The embodiment 140 is connected to the same platform as Foodomat and ReTurno, allowing: unified user identity across all systems, joint loyalty or rewards systems, shared return process for cups, one app interface for orders, pickup, and returns.

The system includes its own user interface 146 in one embodiment. The system features: a touchscreen display for interaction, multilingual support, visual step-by-step guidance, and an option to authenticate via app, card, smartwatch, or phone.

The system includes remote Management & Cloud Connectivity, where administration tasks are completed, including: monitoring inventory levels (beans, milk, water, syrups), adjusting or uploading new drink recipes remotely, reviewing usage analytics, and optimizing stock delivery.

The system includes energy and sustainability features. It includes cup size optimization to reduce waste, an option to disable disposables entirely (reusable-only mode), optional solar/battery operation (for outdoor setups), and water reuse in internal cleaning processes.

The embodiment 140 may be deployed as: a standalone smart beverage kiosk, an integrated module placed adjacent to or embedded within a Foodomat/ReTurno line, or a unified visual and functional unit with shared branding, app, and operational interface.

### Refrigeration Module

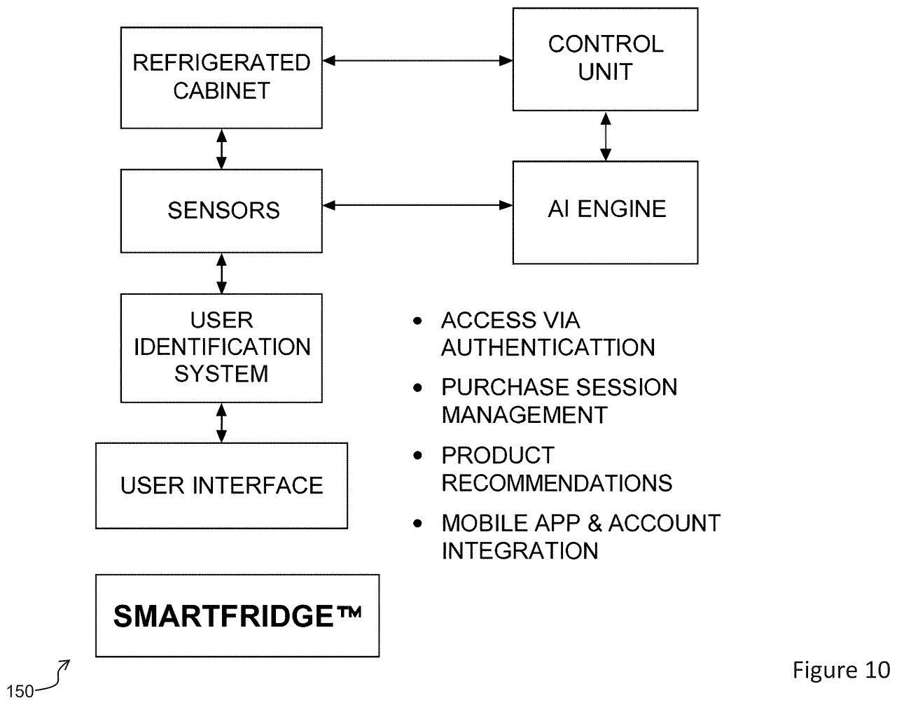

FIG. 10 shows the refrigeration module or SmartFridge embodiment 150.

SmartFridge is an intelligent refrigerated vending unit with transparent doors, designed for dispensing packaged drinks (bottled/canned) and functional food products that complement meals provided by Foodomat and other associated systems. The unit enables personalized access via user identification and uses sensors (cameras, weight, and AI analysis) to detect and charge for selected items. SmartFridge integrates with user profiles, dietary recommendations, and mobile applications, functioning as an extension of the Foodomat, ReTurno, and coffee devices within a unified ecosystem.

The embodiment 150 relates to automated refrigerated vending systems and, more specifically, to intelligent fridges for personalized, health-oriented food and drink delivery, integrated within multi-device food service networks.

Traditional vending fridges lack intelligent personalization, health integration, and seamless connection to broader meal delivery systems. With growing demand for modular, efficient, and eco-conscious food technologies, there is a need for a fridge-based device that adapts to user profiles, dietary goals, and sustainability standards—especially as an extension of modular food delivery ecosystems.

SmartFridge is an advanced refrigerated dispensing unit designed to: offer pre-packaged drinks (bottled/canned) and functional food items tailored to individual needs, use glass doors and internal lighting for product visibility, grant access via wireless identification (NFC/BLE/code), monitor item removal using sensors, cameras, and AI analysis, suggest products based on AI recommendations and health data, integrate with user accounts for automated checkout and nutritional guidance, Operate as a modular and stylistically consistent companion to Foodomat, ReTurno, and coffee modules.

The refrigerated Cabinet includes: an insulated chamber with transparent doors (single or double). It is cooled via a compressor or thermoelectric module (4-10° C.), and it includes internal lighting and shelving for product organization.

User Access is granted via: NFC-enabled devices (smartphone, smartwatch), loyalty cards or employee badges, PIN or QR code via touchscreen/app. Authentication triggers controlled unlocking of fridge doors.

The embodiment 150 includes sensor-based monitoring. Upon door opening, the system activates: weight sensors on each shelf or product zone, AI-powered computer vision (cameras trained to recognize items), door sensors for event timing (start/end of session), and optional RFID readers if products are tagged.

Together, the system detects what is removed, by whom, and when. It includes purchase Session Logic, which operates in one embodiment as follows: first, a door opening initiates a shopping session. The system tracks products taken. When the door closes, the session ends. The user's account is automatically charged for removed items. Receipt and nutritional data are sent to the app.

In one embodiment, the system provides AI-Driven Product Suggestions. During or before access, users receive product suggestions based on preferences, dietary goals, or past choices, suggestions for combos or meal extensions, notifications about allergens, calories, or functional ingredients (e.g., protein, adaptogens, nootropics).

The system includes at least one user Interface. The unit may include: a touchscreen similar to Foodomat and other system modules, real-time product data, pricing, and recommendation display, and multi-language support.

The system includes a mobile app & account Integration. SmartFridge connects with the same app ecosystem as Foodomat: Allows viewing purchase history and receipts, enables remote product browsing and recommendations, tracks health and preference data, supports digital payment or credits.

The module is integrated with the ecosystem. It includes shared infrastructure with Foodomat, a drink dispenser, and ReTurno. Unified identity management and billing, shared maintenance, refilling, and operator backend, consistent design, color scheme, and physical module size.

The smartFridge is designed to be visually consistent with other modular food units, operated within the same digital platform, expandable and replicable across multiple locations, and is adapted to be ideal for workplaces, residential buildings, gyms, and transport hubs.

### Module Interoperation

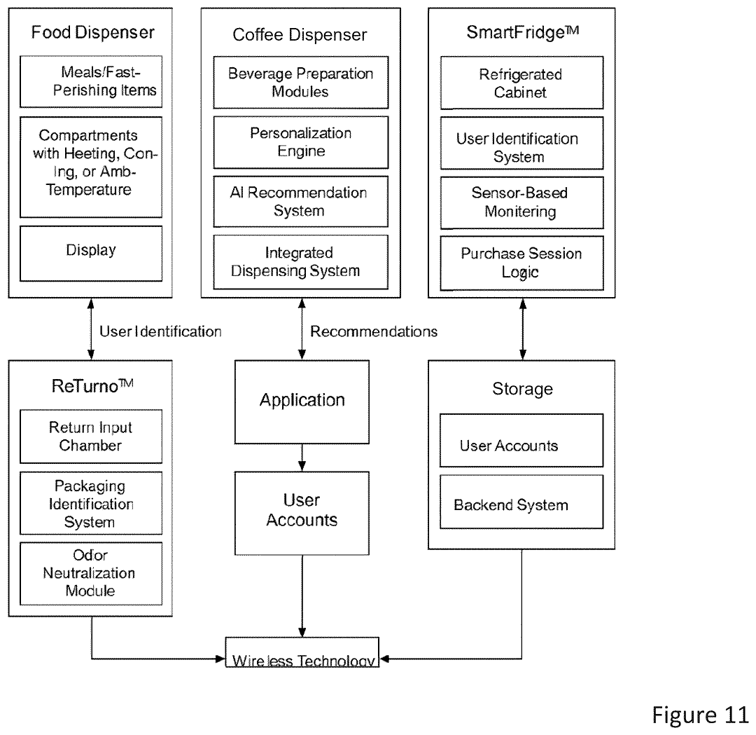

As discussed above and shown in FIG. 11, the various modules are designed to interoperate.

The modularity of the system allows employers, real estate operators, or urban developers to deploy one or multiple modules in tandem, offering an ultra-efficient, data-driven, and sustainable meal distribution platform.

Together, the core Foodomat unit and the ReTurno, Coffee Dispenser, and SmartFridge modules represent a holistic rethinking of how fresh food, drinks, and supplements are delivered, consumed, and cycled in professional and public environments.

All modules share the following traits: seamless software and hardware interoperability. Integration with a common mobile application for ordering, payment, recommendations, and account management. AI personalization engine across all units. Support for reusable packaging, digital receipts, loyalty programs, and ESG tracking. Wireless access technology (NFC/BLE/QR). Optional battery power supply for flexible deployment.

In summary, three exemplary modules were described.

### First, the ReTurno—Automated Packaging Return Station

ReTurno is an intelligent return module for reusable and disposable food packaging. Designed to promote ESG standards and sustainable packaging cycles, ReTurno accepts food containers returned by users after consumption.

Its functionality includes identification of containers using AI-based computer vision, RFID/NFC, barcodes, and/or weight sensors. User authentication via smartphone, NFC card, or wearable device. Automatic registration of refundable deposits (for reusable packaging) or reward points (for single-use compostables). Odor control using ozone modules, cooling systems, or active carbon filtration. Segregated internal bins for multiple container types. Integrated with user profiles and the central app. Fully compatible with Foodomat and other modules.

Second, the coffee module provides Personalized Beverage Dispensing Units. It is an automated drink dispensing machine for hot and cold beverages. It enables users to receive customized drinks, including freshly ground coffee, milk-based drinks, iced drinks, juices, and hot chocolate. Its key features include brewing from fresh beans, ground or instant coffee, and natural or powdered milk, and cold beverage dispensing (juices, cold water, ice, flavored syrups). Personalized recipes retrieved from the user's app profile. AI-driven suggestions based on time, health profile, or consumption history.

The modules provide a Unified user interface with the Foodomat ecosystem. Reusable cup compatibility and integration with ReTurno.

Third, the SmartFridge-Refrigerated Vending Unit for Packaged Products SmartFridge is a refrigerated module with transparent doors, designed for dispensing bottled/canned drinks and functional food products that supplement meals ordered from Foodomats.

The key features include access granted via NFC, QR, mobile app, or wearable device. AI- and sensor-based monitoring system (cameras+weight sensors) detects item removal. Opening the door initiates the shopping session; closing it ends the session and triggers automatic billing. Real-time suggestions and personalization through the app and display are provided. Dispenses personalized functional snacks, supplements, and beverages. Fully integrated with user profiles and Foodomat ordering history. Operates as a cost-optimized, modular extension of the Foodomat system.

In sum, the Foodomat ecosystem-anchored by the core Foodomat unit and extended through the ReTurno, Coffee Dispenser, and SmartFridge modules-delivers a fully integrated, AI-driven solution for fresh food and beverage distribution. Its modular architecture lets organizations deploy only the components they need while ensuring seamless interoperability, unified mobile ordering, and personalized recommendations. By embedding reusable packaging returns, digital payments, and ESG tracking at every touchpoint, the system not only enhances user experience but also advances sustainability goals. Looking forward, this flexible, data-powered platform can scale and adapt to evolving workplace, real estate, and urban environments, establishing a new benchmark for efficient, eco-friendly meal services.

### Custom Packaging

The system also operates with custom packaging.

In one embodiment, the system incorporates specially designed packaging to optimize the freshness of the food and compatibility with the return and sorting module of the distribution points. The packaging is developed with sustainability, hygiene, and automation in mind.

In some embodiments, the custom packaging uses durable materials that withstand repeated use and cleaning cycles. Depending on the application, packaging may be classified as reusable, compostable, or fully recyclable, with each type clearly marked using standardized indicators (e.g., QR codes, RFID tags, or embossed symbols) to enable automated identification and sorting upon return.

Reusable containers and utensils are constructed from high-durability materials such as food-grade stainless steel, silicone, or advanced bioplastics. These containers are designed to be stackable and thermally stable to accommodate both hot and cold food items. They are also engineered with secure sealing mechanisms to prevent leaks during transport and storage in the distribution point. The lids and body components are designed for easy cleaning in industrial washing systems and are resistant to staining or odor retention.

Single-use compostable packaging is manufactured from materials such as molded fiber, PLA (polylactic acid), or bagasse, ensuring rapid breakdown in commercial composting environments. These items are also tagged or printed with eco-certification marks and sorting codes, which the system recognizes to direct them to appropriate bins during the return process.

To facilitate hygiene and safety, all packaging includes a tamper-evident seal and may optionally feature embedded freshness sensors that provide real-time feedback to the data exchange system regarding temperature exposure or seal integrity. The use of smart packaging ensures that the quality of each meal can be verified before distribution, improving transparency and accountability throughout the supply chain.

The packaging system is designed to be closed-loop wherever feasible, reducing the environmental impact associated with food distribution. This integrated packaging model ensures alignment with the system's sustainability goals while maintaining the highest standards of food safety, user convenience, and operational efficiency.

The system shown in the above-captioned figures includes computer logic that allows the depicted embodiments to connect with external services, in some embodiments.

In summary, the technology enables the delivery of functional foods, meaning they improve selected user health deficiencies or goals, e.g., reducing stress, improving memory, boosting immunity, and other benefits.

The technology enables tracking of the supply chain “from farm to fork.”

The technology enables the automation of the meal ordering process by launching AI tools that make decisions related to the dedicated feeding process for the user.

In at least some embodiments, the meal is co-financed not only by the employer, but also by government entities, charities, etc.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

It is to be understood that the above description is intended to be illustrative and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

1. A modular system for distributing perishable items comprising:
at least one distribution point wherein perishable items are brought on-demand to said distribution points by suppliers;
at least one end user interface which allows for purchasing of a perishable item for pickup at a particular distribution point at a particular time; and
a data exchange system that communicates user orders from the end user interface to suppliers and schedules perishable items for pickup at the modular system.

2. A modular food dispensing system, wherein each distribution point comprises a plurality of temperature-controlled lockers, each configured to store perishable items at a designated temperature range.

3. The system of claim 2, wherein each locker includes a thermoelectric heating or cooling module and a digital temperature sensor for environmental monitoring.

4. The system of claim 2, further comprising a touchscreen interface configured to display order information and receive user input for locker access.

5. The system of claim 2, wherein user authentication is performed via wireless technologies selected from the group consisting of NFC, Bluetooth Low Energy (BLE), RFID, and QR code scanning.

6. The system of claim 2, further comprising a local control unit configured to manage locker access, sensor readings, and communication with a remote server.

7. The system of claim 2, wherein the system includes a battery backup module configured to maintain locker operation during power outages.

8. The modular system of claim 1 further comprising a return module, comprising a packaging identification system configured to recognize returned containers using AI-based computer vision, RFID, or barcode scanning.

9. The return module of claim 8, further comprising an odor-neutralization subsystem selected from the group consisting of ozone sterilization, refrigeration, and activated carbon filtration.

10. The return module of claim 8, wherein returned items are associated with specific users via user authentication and logged for reward or deposit tracking.

11. The return module of claim 8, further comprising a contactless input chamber configured to receive and sort returned packaging based on material type.

12. The modular system of claim 1 further comprising a beverage dispensing unit, comprising modules for preparing hot and cold drinks, including espresso, milk-based beverages, juices, and flavored drinks.

13. The beverage dispensing unit of claim 12, wherein drink personalization is performed via a mobile application linked to user profiles.

14. The beverage dispensing unit of claim 12, further comprising an AI-based recommendation engine configured to suggest beverages based on user preferences and health indicators.

15. The beverage dispensing unit of claim 12, wherein the unit is configured to dispense drinks into reusable containers and interface with a return module for container collection.

16. The modular system of claim 1 further comprising a refrigerated vending unit, comprising transparent doors, internal lighting, and sensors configured to detect item removal using weight and computer vision.

17. The refrigerated vending unit of claim 16, wherein user access is granted via wireless identification methods selected from the group consisting of NFC, BLE, and QR code.

18. The refrigerated vending unit of claim 16, further comprising a purchase session logic configured to initiate billing upon door closure and item removal detection.

19. The refrigerated vending unit of claim 16, wherein the unit is integrated with a mobile application for nutritional tracking and personalized product suggestions.

20. A unified food distribution ecosystem, comprising a food dispensing unit, a return module, a beverage dispenser, and a refrigerated vending unit, all interconnected via a centralized mobile application and cloud-based management platform; wherein each module shares user identity management, payment systems, and AI-driven personalization features to deliver a seamless food service experience.
